(19) United States
(12) Patent Application Publication
(10) Pub. No.: US 2026/0346252 A1
(43) Pub. Date: Nov. 5, 2026

(54) MASSIVELY PARALLEL SEQUENTIAL COALESCENCE OF MICRODROPLETS FOR MULTI-STEP REACTIONS

(71) Applicants: The Hong Kong University of Science and Technology, Hong Kong SAR (CN); ADVANCED BIOMEDICAL INSTRUMENTATION CENTRE LIMITED, Hong Kong SAR (CN)

(72) Inventors: Xuyan LIN, Hong Kong SAR (CN); Angela Ruohao WU, Hong Kong SAR (CN); Shuhuai YAO, Hong Kong SAR (CN); Qiangsheng CUI, Hong Kong SAR (CN)

(21) Appl. No.: 19/488,569

(22) PCT Filed: May 31, 2024
(86) PCT No.: PCT/CN2024/096686

§ 371 Date: Nov. 26, 2025

Related U.S. Application Data
(60) Provisional application No. 63/505,695, filed on Jun. 1, 2023.

(51) Int. Cl. B01L 3/00

(52) U.S. Cl. CPC B01L 3/502784; B01L 2200/027; B01L 2200/0647; B01L 2200/16; B01L 2300/0819; B01L 2300/12

(57) ABSTRACT

Microfluidic devices having microwell arrays that enable scalable and parallel droplet multi-step reactions and associated methods for implementing reactions in paired microwells of two different sizes via numerous repeating units of trapping wells as well as sequentially collecting and merging droplets. Droplets that contain the desired species or reagents for reactions are input onto the microfluidic platform. Each well is set up to initially capture one larger droplet, then repeatedly capture smaller ones, followed by merging of larger and smaller droplets. In some embodiments, this action is facilitated by tilting the chip. In some implementations, larger droplets merge three batches of paired, smaller droplets with various needed components in succession while conducting incubation in between the merge and loading. In alternative implementations, each trapping well has two or more active capture sites that merge multiplexed smaller droplets simultaneously and merge more than once.

### CROSS-REFERENCE TO RELATED APPLICATIONS

The present application claims priority to U.S. Provisional Application No. 63/505,695, filed Jun. 1, 2023, the full disclosure of which is incorporated herein by reference in its entirety for all purposes.

### TECHNICAL FIELD

The present disclosure relates to microfluidic devices that facilitate droplet-microfluidics, particularly multi-step reactions, which are useful for a wide range of analytical and bioanalytical applications.

### BACKGROUND

The conventional art includes various approaches and embodiments that facilitate sorting and processing of microdroplets for droplet-microfluidics, including those described in patent publications: WO 2016/149661A1; WO 2019/136058A1; U.S. Pat. Nos. 10,543,485; 11,358,105; US 2019/0127782A1; EP3299469B 1 and various related research publications.

The embodiments described in patent WO 2016/149661A1, correspond to a 2019 research publication entitled “Massively Parallel Screening of Synthetic Microbial Communities” by Kehe et al., which describes microwells of uniform size and shape designed to contain circular-shaped droplets that overlap. All droplets containing different reactants are of the same size and are mixed before being randomly paired with each other after being trapped in the microwells. The droplets are introduced into the microfluidic chip through a loading slot and are trapped in the microwells by buoyancy. As demonstrated in the publication, the microwell chip (kChip) is equipped with a PMMA cassette comprising holders, spacers, and magnets, into which fluorinated oil is injected for flushing the droplets. The publication notes this chip is suitable for screening synthetic microbial communities, detecting various viral and bacterial pathogens, and discovering novel microbial interactions.

WO2019/136058A1 by Chung et al. describes microfluidic chips configured for droplet generation, sorting, and droplet capture microwells. The microwells are circular in shape and connected to U-shaped wells, with the circular wells being deeper than the U-shaped ones. The droplets are captured by buoyancy and can be merged using demulsifiers or an electric field. The merged droplets can then be used for single-cell sequencing library preparation or RT-LAMP reactions.

Various other research publications by others describe alternative approaches, including Xie et al. (“Combinatorial perturbation sequencing on single cells using microwell-based droplet random pairing,” 2022) and Wu et al. (“CoID-LAMP: Color-Encoded, Intelligent Digital LAMP for Multiplex Nucleic Acid Quantification,” 2023), which describe microwells with similar features as those developed by Chung et al. (“Single-cell RT-LAMP mRNA detection by integrated droplet sorting and merging” 2029). In the work by Xie, the microwells are circular in shape and connected to U-shaped wells of the same depth, trapping one larger droplet and two smaller droplets at a time. The droplets in these microwells can be randomly paired, enabling the identification of rare cell populations and the discovery of novel genetic interactions at the single-cell level. In the work by Wu et al., the circular part of the microwell is deeper than the U-shaped part, with the circular part capturing one larger droplet and the U-shaped part capturing a smaller droplet. The captured droplets can be merged using a corona treater, and the microwell-based droplets are capable of highly sensitive and specific detection of multiple nucleic acid targets simultaneously. In other research by Cai et al. (“Droplet Encoding-Pairing Enabled Multiplexed Digital Loop-Mediated Isothermal Amplification for Simultaneous Quantitative Detection of Multiple Pathogens,” 2023), droplets of two distinct sizes are captured by calabash-shaped microwells consisting of two cylindrical structures at two different heights. The droplets are floated into the larger sub-wells by buoyancy force, and the droplet encoding-pairing approach in the chip is used for simultaneous quantitative detection of multiple pathogens.

US2019/0127782A1 and EP3299469B1 utilize droplets for high-throughput RNA sequencing, called inDrop and Drop-seq, respectively, which generates each droplet that comprises a single cell, lysis buffer, and barcode bead. The droplets are then collected in a PCR tube for further incubation and reactions, which is a single-step on-chip reaction and is suitable for single omics sequencing.

None of the patents or literature mentioned above have performed reactions of more than two steps. Apart from patent WO2016/149661A1 and its related literature, where larger droplets are spread in microwells first, followed by loading of smaller droplets, the reactions in these works are conducted by adding reactants to the droplets in single microwells that are merged once. The sizes of the connecting trapping wells are not designed for multi-step reactions, and there is no suggestion of controlling to a precise ratio of the two distinct sizes of droplets for reactions over multiple steps.

The patents and literature mentioned above describe working principles that differ from the inventive concepts described herein. U.S. Pat. No. 10,543,485 and work by Yu et al. (“Parallel multistep digital analysis SlipChip demonstrated with the quantification of nucleic acid by digital LAMP-CRISPR,” 2022) utilize SlipChip technology, which involves use of two plates that move relative to each other to open or close channels for fluid flow. This allows for precise control of reactions, such as separating different samples, mixing them, and carrying out reactions within individual chambers. However, the complex chip design and fabrication process may limit its throughput and widespread use. Another technique for achieving multi-step reactions is pico-injection, which is illustrated in U.S. Pat. No. 11,358,105 and work by Zheng et al (“High-throughput, single-microbe genomics with strain resolution, applied to a human gut microbiome,” 2022). This method involves using a microfabricated channel with a narrow constriction that serves as a nozzle for injecting substances into single droplets by applying a high-voltage pulse. While this technique can be used for library preparation for single-cell sequencing, in-tube collection, and reinjection may lead to sample loss after each incubation step, and it requires complicated apparatus and chip design.

Thus, each of the above described conventional approaches has significant drawbacks, limitations, and challenges to address. Thus, there remains a need for microfluidic devices with microarrays that facilitate multi-step reactions. There is further need for such microfluidic device that allow for repeated steps of loading and merging droplets of differing sizes to facilitate multi-step reactions. There is still further need for microfluidic devices that support coalescence between droplets of particular differing dimensions and/or ratios. There is further need for such devices that are amenable to simplicity in fabrication and ease of use.

### SUMMARY

The embodiments described herein include systems, methods, design parameters, and applications related to microfluidic devices having microwell arrays that enable scalable and parallel droplet multi-step reactions and associated methods of use.

In one aspect, the microwells in this design are specifically configured to capture individual droplets of at least two distinct sizes. The larger droplet, also known as the first droplet, is trapped in a circular-shaped well, while at least one smaller droplet, referred to as the second or subsequent droplet, is captured in a U-shaped well in the presence of the first droplet. In some embodiments, the ratio of the diameter of the circular trapping site to the U-shaped trapping site falls between 1.25-1.75:1, with an overlap of two trapping sides in diameters ranging from 30%-10%. Furthermore, the size ratio of the larger droplets to smaller droplets can vary between 1.5-1.9:1, depending on the number of sequential steps of droplet input. By this approach, the design allows for performing multi-step reactions in one microwell array chip. In some embodiments, the first droplet includes biological cells and the second droplet includes one or more reagents. In some embodiments, the first droplet includes one or more reagents and the second droplet includes biological cells. It is appreciated that the terms “first” and “second” are for identification purposes and do not limit these concepts to a specific order. In some embodiments, the first plurality of droplets are delivered before the second plurality of droplets, whereas in other embodiments, the first plurality of droplets are delivered after the second plurality of droplets.

In contrast to the conventional approaches described in the background, the invention involves the utilization of a droplet microfluidic platform to perform high-throughput reactions. This microfluidic device can include an array of microwells, with each well containing two fluidically connected trapping sites of varying sizes. The microwell's trapping sites are designed to capture stepwise droplets of two different sizes based on their respective sizes. By conducting repetitive loading-merge steps, multi-step reactions can be carried out simultaneously in tens of thousands of droplets in parallel. The coalescence step for all droplets in the on-chip microwell array can be executed simultaneously within a matter of seconds (e.g. 10 seconds or less, 5 seconds or less, 3 seconds or less), in a highly parallel manner. In some embodiments, the number of reactions is over twice that provided by conventional approaches. The limitation of reactions depends on the designation of overlap of the two trapping sites. In an exemplary embodiment, the approach provides four or more, typically four reactions, with time per loading being within 3-15 minutes, typically between 6-10 minutes per loading. Our approach offers several advantages, including reduced time consumption, minimized sample loss, and simplified apparatus, making it a valuable tool for a wide range of applications in high-throughput screening and analysis. In some embodiments, the microarray design described herein allows on-chip incubation and the addition of different reagents according to the reaction protocol, offering more possibilities for multi-omics sequencing. These innovations simplify the process of creating a standard operating protocol (SOP) and chip design for performing high-throughput multi-step multi-omics sequencing or other multi-step reactions using microdroplets. The microfluidic device and microarray concepts described herein are ideal for laboratory settings and can generate multi-omic single-cell data from rare cells and frozen patient tumor samples, with the capability of analyzing more than 10,000 cells at a time.

In another aspect, the embodiments described herein provide design features and parameters that implement reactions in paired microwells of two different sizes via numerous repeating units of trapping wells as well as sequentially collecting and merging droplets. Droplets that contain the desired species (such as cells for example) or reagents for reactions are input onto the microfluidic platform. Each well is configured to initially capture one larger droplet, then to repeatedly capture smaller droplets while tilting the chip, followed by the merging of larger and smaller droplets. According to some implementations, the larger droplets can merge three batches of paired, smaller droplets with various needed components in succession while conducting incubation in the time between the merge and loading. This capability is demonstrated specifically through applying multi-step reactions for highly parallel multi-omics profiling. In alternative implementations, each trapping well has two or more active capture sites that can merge multiplexed smaller droplets simultaneously and merge the droplets more than once.

### BRIEF DESCRIPTION OF DRAWINGS

FIG. 1A shows a schematic representation of a microwell array chip device employed for capturing droplets and merging them sequentially in accordance with some embodiments, in accordance with some embodiments; FIG. 1B shows a substrate utilized for the microwell array chip, in accordance with some embodiments.

FIG. 2A illustrates loading of larger droplets and smaller droplets into the microwells of an exemplary device, in accordance with some embodiments; FIG. 2B displays droplets with two distinct diameters being trapped in the microwells, in accordance with some embodiments; and FIG. 2C demonstrates the previously designed calabash patterns for multi-step reactions, in accordance with some embodiments.

FIG. 3A illustrates a microwell comprising a circular well connected to two U-shaped wells, in accordance with some embodiments; FIG. 3B illustrates a microwell that includes a circular well connected to three U-shaped wells, in accordance with some embodiments; and FIG. 3C illustrates a microwell including a circular well connected to four U-shaped wells, in accordance with some embodiments.

FIG. 4 presents a schematic representation of an example of the manufacturing components of a microfluidic device, in accordance with some embodiments.

FIGS. 5A-5C show top view schematics demonstrating the process of loading droplets with different sizes through the flowing channel to the microwells, in accordance with some embodiments; FIG. 5A shows the loading of larger droplets onto the input of the microfluidic device's flow channel, in accordance with some embodiments; FIG. 5B shows the process of trapping the larger droplets into the microwells by tilting, in accordance with some embodiments. FIG. 5C shows the larger droplets being loaded into the microwells after occupying the circular trapping sites of the microwells, also achieved by tilting, in accordance with some embodiments.

FIG. 6A depicts an exemplary embodiment of a single trapping well featuring a microwell that includes a circular trapping site and a U-shaped trapping site for trapping different sized droplets, in accordance with some embodiments; FIG. 6B displays two droplets in an exemplary trapping well with a precise ratio of the diameter of the larger droplet to the smaller droplet set, in accordance with some embodiments.

FIG. 7A displays an image of a microwell array chip device ready for use, in accordance with some embodiments; FIG. 7B depicts the CAD layout of a top-down view of an exemplary microwell array chip device that includes an array of microwells, each having a circular trapping site and a U-shaped trapping site, in accordance with some embodiments; FIG. 7C illustrates a side view of the exemplary device, which includes a flow channel layer situated on the substrate below the microwell array, in accordance with some embodiments; FIG. 7D portrays the primary view of the exemplary device, which comprises a cover, a microwell array slab, and a substrate, in accordance with some embodiments.

FIG. 8 presents a schematic illustration of the multi-step reaction via sequential droplet addition facilitated by a specialized microwell, in accordance with some embodiments.

FIGS. 9A-9H exhibit a step-by-step schematic (top view) and images (top view) of a multi-step reaction process via sequential droplet addition in a microwell array chip, in accordance with some embodiments.

FIG. 10 shows a flowchart of an exemplary method of performing multi-step reactions using a microfluidic device in accordance with some embodiments.

### DETAILED DESCRIPTION

The invention pertains to systems, methods, design parameters, and devices that facilitate droplet-microfluidics, particularly multi-step reactions, which are useful for a wide range of analytical and bioanalytical applications.

### 1. Overview of the Invention

As described herein, the invention pertains to systems, methods, design parameters, and applications related to microfluidic devices having microwell arrays that enable scalable and parallel droplet multi-step reactions and methods of using such devices. The embodiments provide design parameters that facilitate implementing reactions in paired microwells of two different sizes via numerous repeating units of trapping wells as well as sequentially collecting and merging droplets. Droplets that contain the desired species (such as cells for example) or reagents for reactions are put onto the microfluidic platform. Each well is set up to initially capture one larger droplet, then to repeatedly capture smaller ones while tilting the chip, followed by the merging of larger and smaller droplets. According to a particular implementation, the larger droplets can merge three batches of paired, smaller droplets with various needed components in succession while conducting incubation in the time between the merge and loading. This is demonstrated specifically through applying multi-step reactions for highly parallel multi-omics profiling. In alternative implementations, each trapping well has two or more active capture sites that can merge multiplexed smaller droplets simultaneously and that can merge them more than once.

In an exemplary embodiment, single cells with lysis buffer are encapsulated within larger droplets and deposited in a circular portion of each microwell. Subsequently, reagents used for performing DNA fragmentation, barcoding, and RNA reverse transcription are generated in smaller droplets that are trapped in U-shaped portions of each microwell and merged sequentially with the larger droplets containing the single cells in the circular trapping sites. This enables the single cells deposited in each microwell to undergo multi-step reactions sequentially, including cell lysis, DNA fragmentation, DNA/RNA barcoding, and generating complementary DNA (cDNA) from RNA. The optimal reaction temperatures can be achieved using a hotplate or PCR machine with a flat stage or any suitable thermal control device.

### 2. Properties of the Microfluidic Device

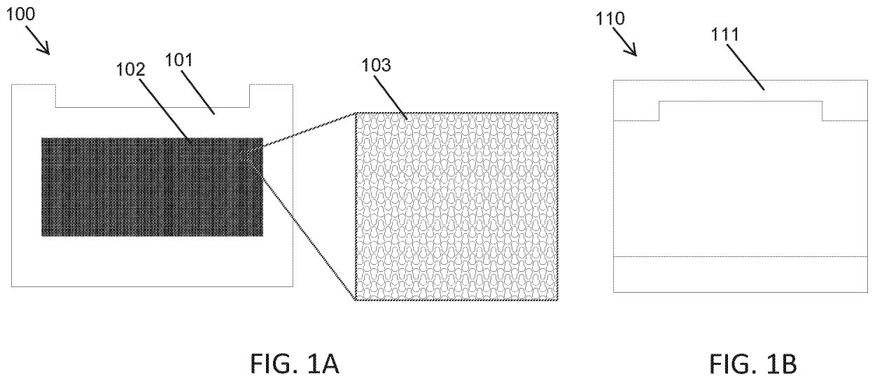

A microfluidic device includes the following aspects/features: a) chip design b) whole chip device design (e.g. Polydimethylsiloxane (PDMS) chip, glass cover, and substrate); c) droplet manipulation features; and d) well combination and droplet volume design parameters. These aspects allow for the advantageous capabilities described herein. In an exemplary embodiment, the microfluidic device microfluidic devices comprising a microwell with at least one input for bigger droplets and one input for smaller droplets, at least one loading channel, and a slab comprising a number of trapping sites within a microwell array (see for example, embodiments in FIGS. 1A-1B).

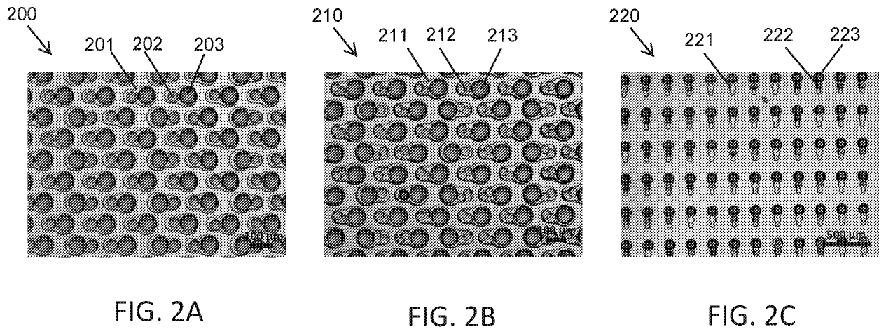

The microfluidic systems described in various embodiments include microwells that act as capture wells that include at least two fluidically connected trapping sites in two distinct sizes. Some embodiments may include additional trapping sites of same or different sizes. Some embodiments may include trapping sites with same or different depths. Each microwell is designed to have a larger trapping shape (e.g. circular) to trap a single first larger droplet and at least one smaller trapping shape (e.g. U-shape or smaller rounded shape) sized to trap one second or subsequent smaller droplet when the first larger droplet is present in the trapping site (see for example, FIG. 2A). In some embodiment, the diameter ratio of the larger (e.g. circular) shaped trapping site to the smaller (e.g. U-shaped) trapping site ranges from about 1.25-1.75:1, with an overlap of two trapping sides in diameters is about 30%-10%. Additionally, the ratio of the diameter of larger droplets to smaller droplets is within a range of about 1.5-1.9:1, which varies depending on the number of sequential input droplets. Deviations from this diameter ratio range may lead to low occupancy of the second droplet or smaller (e.g. U-shaped) trapping sites containing two smaller droplets in each well (see embodiment in FIG. 2B). An inadequately designed trapping site for subsequent droplets may result in a decreased rate of successful capture of smaller droplets (see embodiment in FIG. 2C). In some embodiments, the microwells are designed to trap a plurality of first droplets and a plurality of second or subsequent droplets, where the first droplets are larger than the second or subsequent droplets by a certain percentage (e.g., 20%, 30%, 40%, 50%, 60% , 70% or more). In some embodiments, the overlap between larger/smaller trapping sites is designed to facilitate merging of larger and smaller droplets disposed in the larger/smaller trapping sites. The overlap can be within a range of 5% to 50%, typically 10%-30%, more typically about 20%.

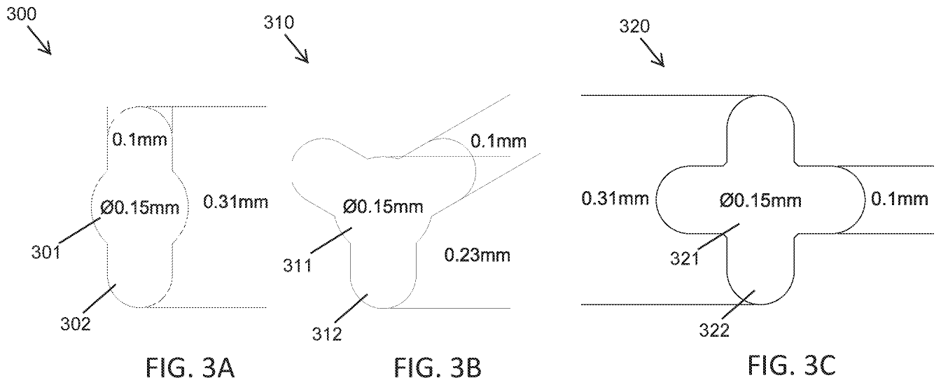

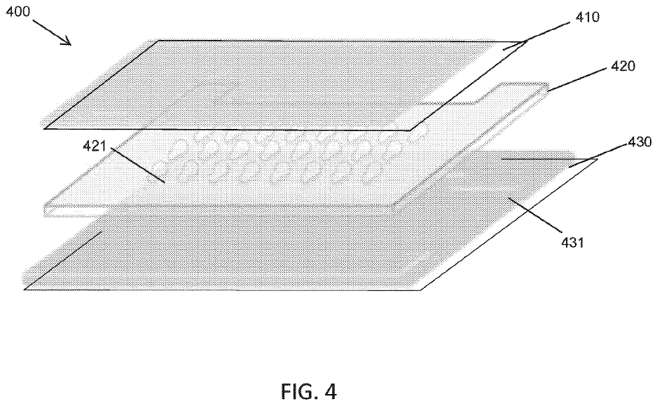

In some embodiments, the microfluidic device includes an array of microwells, where each microwell includes a circular well and a U-shaped well, with each trapping well designed to hold one larger droplet and one smaller droplet. Alternatively, in other embodiments, each microwell includes a circular well connected to two, three, four or more U-shaped wells, with each trapping site intended to capture one larger droplet followed by simultaneously capturing two, three, or four smaller droplets, respectively (see embodiments in FIGS. 3A-3C). In some embodiments, each microwell of the microfluidic chip array has a circular trapping site with a diameter ranging from 100 to 600 μm, and one or more U-shaped trapping sites, each with a diameter ranging from 57 to 120 μm and a depth of 80 to 130 μm. In some embodiments, the circular well has the same depth as the U-shaped well, while in other embodiments, the circular well is deeper. In some embodiments, the microfluidic chip is a slab that includes multiple microwells, and the size of the slab can range from a few microns to 70 millimeters in width or length and from a few microns to 1 cm in thickness. The number and density of the microwells can vary based on the desired application, as well as the size and volume of the factors being encapsulated, the reaction type, and the detection technique. These inventive concepts are not limited by the number of microwells, and a large number of wells can be incorporated into the microfluidic devices. In some embodiments, the microfluidic devices can have from about 100 to 90,000 microwells arranged in columns and rows, with some embodiments having perpendicular X-Y grids, while others have non-perpendicular orientations of columns and rows (e.g. staggered or interleaved rows). In some embodiments, the irregular shape of the microwells allow the microwells to be fit together in a staggered or interleaved pattern to increase density of microwells. In some embodiments, a glass cover is affixed to the backside of the microwell slab in order to provide additional structural reinforcement to the silicone-based microfluidic device and to minimize evaporation (see the components of an exemplary assembly in FIG. 4).

In some embodiments, the microwell array is positioned above or below the flow channel layer to ensure that each microwell within the array can be reached through the flow channel. In certain embodiments, the devices include a single flow channel, while in other embodiments, the devices can feature two or more flow channels. The flow channel can be defined on either the top or bottom layer of the devices. In one embodiment, the flow channel is created by mounting a solid substrate that serves as a spacer for the microwell array.

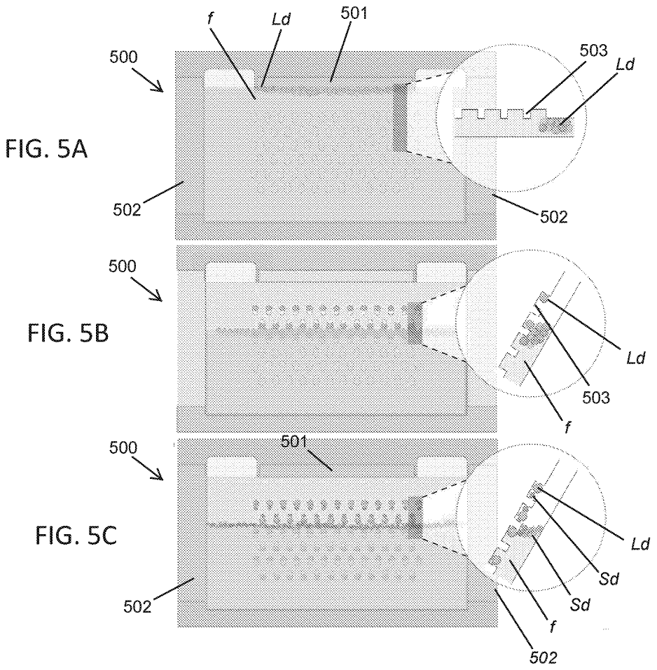

Alternatively, the flow channel can be incorporated into the same mold that is utilized to define the microwell array. Droplets sink and feed by a combination of buoyancy and capillary force from one or more flow channels into empty microwell spaces while tilting the chip (as described in detail in FIGS. 5A-5C). In some embodiments, the microfluidic device is inverted to allow the merged droplets to enter the flow channel and flow toward the output.

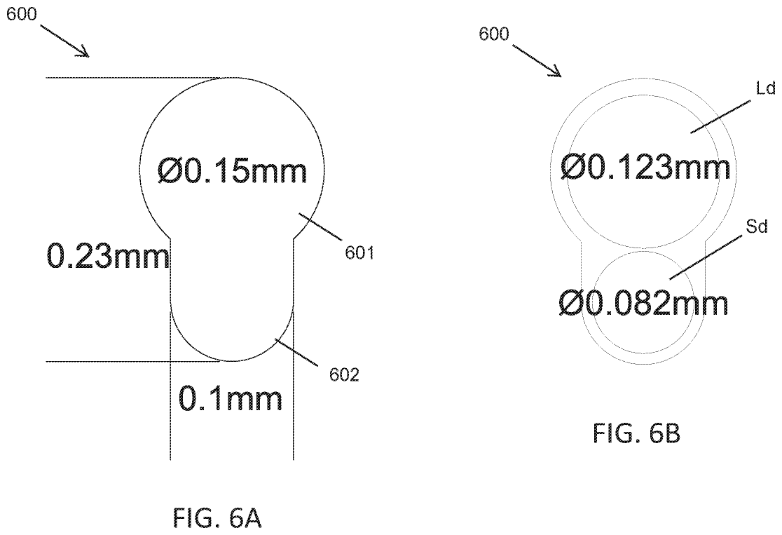

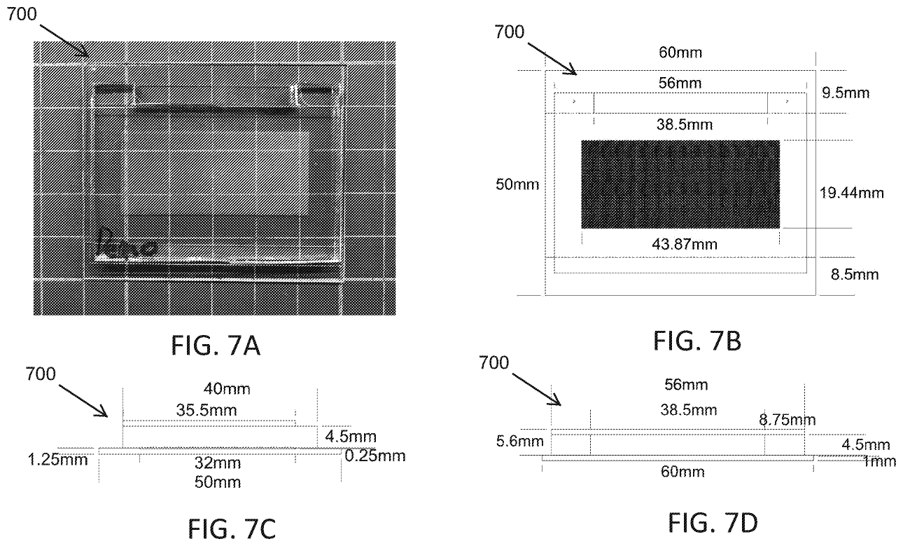

In certain example embodiments, the microwell is designed with a circular trapping site and a U-shape trapping site at a diameter 150 μm and 100 μm respectively, with 15% overlap of two trapping sites in diameters (as shown in the embodiment of FIG. 6A). The diameter of the first droplet, which is larger, is approximately 120-135 μm, while the size of the second and subsequent droplets, which are smaller, ranges from about 85 to 70 μm (see embodiment in FIG. 6B). The single flow channel is located above the microwell array, which is situated in the cavity of the glass substrate with a depth of 255 μm (see FIG. 7A).

### 3. Performance of the Microfluidic Device

Droplets of two distinct sizes, each containing different molecular species or reagents, are generated and loaded onto the chip. These droplets can be formed outside of the chip and subsequently loaded onto the microwell array chip through the droplet entrance. Alternatively, the droplets can be generated within a microfluidic chip that is directly connected to the microfluidic devices featuring the microwell array. This allows the droplets to be fed into the chip while forming. Certain embodiments exemplify the generation of droplets in the off-chip droplet generation modules, followed by their loading onto the input.

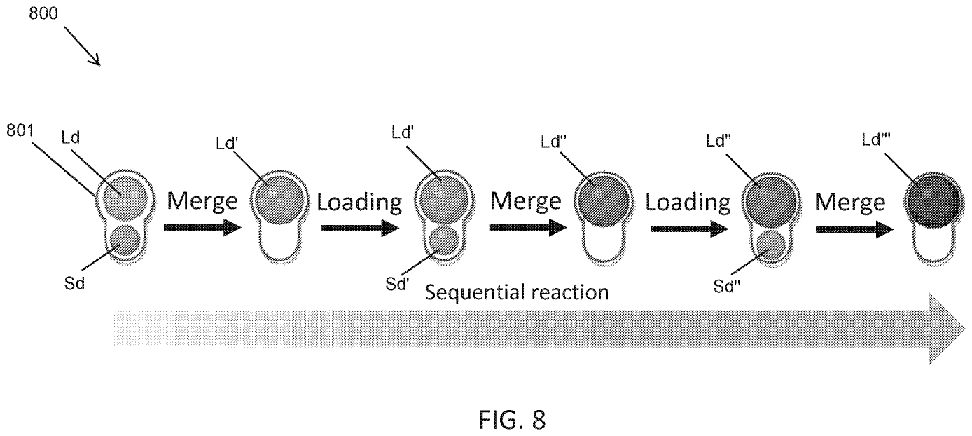

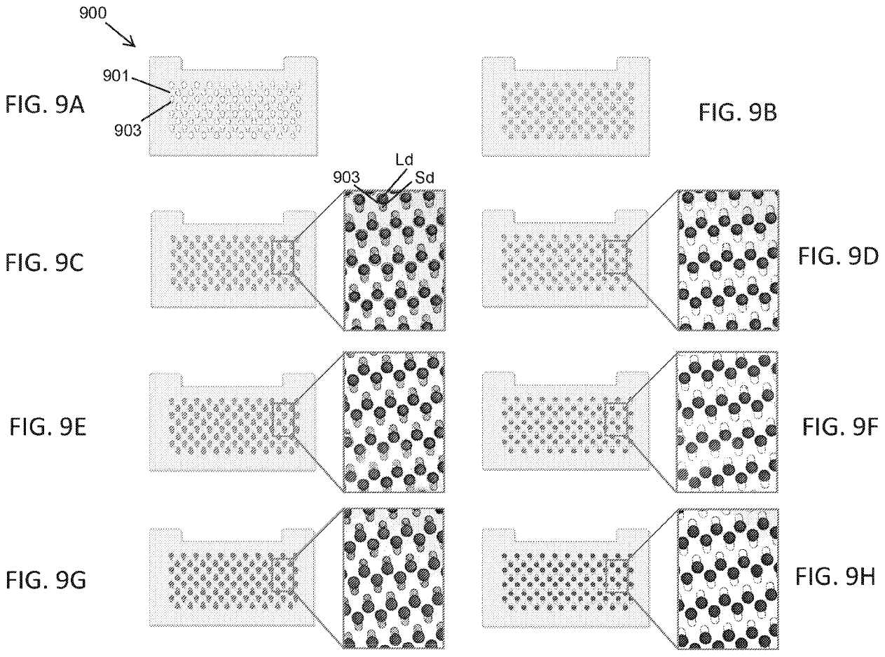

These droplets are subsequently dispersed throughout the flow channels of the microfluidic devices for transportation to the microwells in accordance with their respective sizes. The microwells have been dimensioned to contain a specific quantity of discrete droplets that can coalesce into a single droplet, thus enabling the merging of the contents of the individual droplets. The second (smaller) droplets are absorbed into the first (larger) droplet, where vacating the U-shaped wells allows subsequent droplets to occupy followed by sequentially merging (see example in FIG. 8). The sizes of the droplets, both larger and smaller, need to be carefully designed and calculated to ensure that they fit within the microwells before and after merging. In certain example embodiments, the larger droplet is loaded onto the input of the chip and is captured by the circular shape trapping sites of the microwells when tilting the chip. Similarly, the second droplets in smaller sizes enter the U-shape trapping sites. After the first merging step, the smaller droplet is absorbed into the larger droplet, leaving the smaller well empty for the subsequent merging step (see FIG. 9). This process can be repeated for at least two more merging steps to reach the massively multi-step reaction.

Paired droplets distributed in microwells coalescence in parallel through various mechanisms that merge two or more paired droplets into a single droplet. These mechanisms include demulsification, electrocoalescence, thermal coalescence, acoustic coalescence, among others. In the certain example embodiment, the droplets are merged by employing a demulsifier, which depletes the surfactant concentration of the droplets.

### 4. Applicability for Different Fabrications

The microfluidic microwell array device described herein may be a silicone-based chip and can be fabricated using various standard microfabrication techniques, including but not limited to photolithography, soft lithography, microcontact printing, hot embossing, injection molding, LIGA. Suitable materials can be used for fabricating microfluidic devices, including but not limit to glass, silicon, or polycarbonate (PC), poly(dimethylsiloxane) (PDMS), poly(methylacrylate) (PMMA), polystyrene (PS), cyclic olefin copolymer (COC). Microfluidic devices can be made from a variety of materials, including glass, silicon, polycarbonate (PC), poly(dimethylsiloxane) (PDMS), poly(methylacrylate) (PMMA), polystyrene (PS), and cyclic olefin copolymer (COC). The microfluidic flow channels can be created by etching the substrate or using a mold to make a microchannel structure. In one embodiment, PDMS may be used to prepare microfluidic devices through soft lithography. For instance, a mold may be produced by photolithography to define the location and dimensions of the microwell array. The substrate material can be poured into the mold to make a stamp that is then bonded to the glass substrate.

In some embodiments, it may be desirable to reduce the chance of reactants interacting with the inner surfaces of the microwells due to the surface properties of certain polymers. To achieve this, passivation treatment may be necessary. The microwells can be coated by adding reagents or modifying the substrate surface to prevent interaction between droplets and surface. Depending on the properties of the reactant, hydrophobic or hydrophilic coating can be preferred. Surface treatment reagents, such as silanes, Parylene, surfactants, or specific proteins or peptides, can be used for this purpose in some embodiments.

### 5. Demonstration on Single-Cell Sequencing Library Construction (Cell Lysis, Barcoding, and DNA-RNA Co-Amplification)

As described herein, the present invention relates to methods for high-throughput, multi-step reactions of molecular species. These methods can be used to combine and profile any pre-defined molecular species. One example involves a sample contained in a droplet that is captured into a trapping site based on its size and stepwise reagent. For instance, a sample containing distinct nucleic acids can be captured in larger droplets presented in a trapping site of a microwell first, while downstream reagents can be contained in smaller droplets that are trapped in the other site of the same microwell sequentially. Other embodiments involve the use of single cells or cell lysates as the sample and various combinations of enzymes, primers, and buffers as downstream reagents.

In some embodiments, a segment of a primer can function as a unique molecular identifier (UMI) that is provided to a microwell via a droplet, allowing for the identification or differentiation of the reaction compartment. Additionally, a primer may contain one or more barcoding sequences that allow for the identification of the source of an amplified product nucleic acid. Barcodes can take various forms, such as oligonucleotides, hydrogel beads synthesized with primers and barcodes in a split-and-pool manner, or colorimetric or fluorescent dye-containing species droplets. The barcode may also be microwell-denoted to differentiate the reaction in diverse microwells for observation or imaging.

The droplets can contain a variety of biomolecules or species, such as single cells, nucleic acid, proteins, or antibodies depending on the application including sequencing library construction, gene expression analysis, protein-protein interaction studies, and drug screening. The reaction sites can be designed selectively to a target biomolecule in the sample, such as a specific DNA sequence or a protein of interest, coalescence with the desired reagents. The reaction conditions, such as temperature, pH, and concentration of reagents, can be controlled by the microfluidic system to optimize the reaction.

In an exemplary embodiment, single cells with lysis buffer are encapsulated within larger droplets and deposited in a circular portion of each microwell. Subsequently, reagents used for performing polyA polymerase, barcoding, and RNA reverse transcription are generated in smaller droplets that are trapped in the U-shape of each microwell and merged sequentially with the larger droplets containing the single cells in the circular trapping sites. This enables the single cells deposited in each microwell to undergo multi-step reactions sequentially, including but not limited to cell lysis, adding polyA tail for total RNA, RNA barcoding, generating complementary DNA (cDNA) from RNA, and others. The optimal reaction temperatures can be achieved using a hotplate or PCR machine with a flat stage.

### 6. Exemplary Embodiments

The above features can be further understood by referring to the embodiments in FIGS. 1A-10, which are described in further detail below.

FIG. 1A shows a schematic representation of a microwell array chip device 100 employed for capturing droplets and merging them sequentially in accordance with some embodiments. Chip device 100 includes a plate 101 having an interior region 102 in which a microarray with specially shaped wells 103 is defined to facilitate the microfluidic action and multi-step reactions described herein; FIG. 1B shows a substrate 110 utilized for the microwell array chip 100, which features a groove 111 serving as a flow channel for loading droplets into the chip device 100. As described further below, the wells 103 are specially shaped to trap droplets of particular differing sizes.

FIGS. 2A-2C illustrate exemplary embodiments of the specialized well designs that trap droplets of particular differing sizes. FIG. 2A shows an exemplary microwell design 200 with microwells 201 shaped and arranged to facilitate the loading of larger droplets 203 with a diameter ratio of 1.6:1 to smaller droplets 202 into the microwells 201, resulting in a 100% capture rate; FIG. 2B shows an exemplary microwell design 210 with microwells 211 shaped and arranged to facilitate loading of droplets with two distinct diameters (larger droplets 213 with smaller droplets 212) at a ratio of 2.1:1 being trapped in the microwells 211, where 92% of the U-shaped trapping sites contained two smaller droplets in each well; and FIG. 2C shows yet another microwell design 220 that demonstrates the previously designed calabash patterns of microwells 221 for multi-step reactions, which showed an occupancy rate of 61% for smaller droplets 222 in the second calabash trapping sites and almost 100% for larger droplets 223 in the first trapping site of the microwell 221. As can be seen above in FIGS. 2A-2B, the microwells can be disposed in an alternating orientations with rows being interleaved to increase microwell density in the chip. As can be seen in FIG. 2C, these microwells are disposed in a same orientation and disposed in a regular rows and columns. It is appreciated that any of the microwell shapes described herein can be disposed in any number of patterns including but not limited to: orientations that alternative in two or more directions, staggered rows, aligned orientations, regular spaced rows. While the above described designs are configured to trap droplets of a particular size and ratio, it is appreciated that the microwells could be designed to accommodate various other droplet sizes and ratios as well, as needed for a particular application.

FIGS. 3A-3C illustrate another exemplary design of microwells (300, 310, 320). In such embodiments, the microwells are shaped to feature a circular trapping site (301, 311, 321) along with one or more U-shaped trapping sites (302, 312, 322) connected thereto, the circular trapping site designed to trap a larger droplet and the one or more U-shaped trapping sites designed and sized to trap smaller droplets. In an exemplary embodiment, the circular trapping site has a diameter within a range of 125-200 μm (e.g. 150 μm) and one or more U-shaped trapping sites with a diameter of less than 125 μm, such as within a range of 50 μm to 125 μm (e.g. 100 μm). It is appreciated that the “circular” trapping site is not required to be a circle and can be of any configuration to trap a circular droplet of a set size (for example, the microwell of FIG. 3C). FIG. 3A illustrates a microwell 300 comprising a circular well 301 connected to two U-shaped wells 302 on opposite sides, where each trapping site is designed to capture one larger droplet followed by simultaneously capturing two smaller droplets; FIG. 3B displays a microwell 310 that includes a circular well 311 connected to three U-shaped wells 312, where each trapping site is intended to capture one larger droplet followed by simultaneously capturing three smaller droplets; and FIG. 3C presents a microwell 320 including a circular trapping well 321 connected to four U-shaped wells 322, where the “circular” well trapping site is intended to capture one larger droplet followed by simultaneously capturing four smaller droplets in the U-shaped wells. While the U-shaped wells are shown at particular orientations and placements, other variations could be realized. Further, the microwell depicted are shown as having particular dimensions, and it is understood that these concepts are not limited to these dimensions and could be modified and sized to trap droplets of various other sizes accordingly.

FIG. 4 presents a schematic representation of an example of the manufacturing components of the microfluidic devices disclosed herein. In this exploded view, an exemplary chip device 400 include a cover 410 disposed atop a chip 410 having defined therein an array of microwells 421, such as any of those described herein, and a substrate 430 that supports the chip layer. In some embodiments, the substrate layer 430 may include one or more grooves or channels 431 defined therein that when attached to the chip layer form the fluid paths through the microfluidic device.

FIGS. 5A-5C show top view schematics demonstrating the process of loading droplets with different sizes through the flowing channel to the microwells, as seen from the side view inserts; FIG. 5A shows the loading of larger droplets Ld through the input 501 at the top of the microfluidic device 500 flow channel via flow of a carrier fluid f. The carrier fluid can include one or more fluids such as FC-40, HFE-7500, HFE-7100 and other fluids with higher densities than water that are commonly used in microfluidics. Outlets 502 at the sides of the chip allow excess carrier fluid to flow out of the chip flow channel after loading. The fluid f carries the large droplets Ld which are transmitted by fluid flow through the fluid channel and across the microarray having an array of microwells 503 defined therein, as can be seen in the enlarged side view insert at right; FIG. 5B illustrates the process of trapping the larger droplets Ld into the microwells 503 by tilting the microfluidic device 500. As can be seen in the side view insert, as the fluid f recedes due to tilting, the larger droplets engage within correspondingly sized microwells 503. FIG. 5C depicts the larger droplets being loaded into the microwells after occupying the circular trapping sites of the microwells with the smaller droplets Sd, which can be facilitate by smaller dimensioned portions (e.g. U-shaped portions) of the microwells, also achieved by tilting. As can be seen in the side view insert, as the fluid f recedes due to tilting, the smaller droplets engage within correspondingly sized portions of the microwells 503. In some embodiments, the circular trapping site and the U-shaped portions of the microwell are the same or substantially the same depth, as can been appreciated in the side view insert of FIGS. 5A-5C. In this embodiment, the large and small droplets are delivered separately by separate carrier fluids.

FIG. 6A depicts an exemplary embodiment of a single trapping well featuring a microwell 600 that includes a circular trapping site 601 and a U-shaped trapping site 602. In this embodiment, the circular trapping site has a diameter of 150 μm and the U-shaped site has a diameter of 100 μm, respectively. Further, in this embodiment, there is about 20% overlap of the two trapping sites in diameter; FIG. 6B displays two droplets Ld and Sd in trapping well 600 configured such that a ratio of the diameter of the larger droplet to the smaller droplet set at 1.5. This demonstrates how the larger and smaller droplets can be loaded efficiently within the microwell. While certain dimensions are depicted here for droplets of a particular size and ratio between sizes, it is appreciated that various other dimensions could be used in order to facilitate trapping of droplets of various other sizes and ratio differentials.

FIG. 7A displays an image of a microwell array chip device 700 ready for use, as another example. As shown, the microwell array chip device can be formed of transparent or translucent materials such that the trapping of droplets can be readily detected, observed and confirmed. FIG. 7B depicts the CAD layout of a top-down view of an exemplary embodiment featuring a microfluidic microwell array chip device 700 having an array of microwells, each having a circular trapping site with a diameter of 150 μm and a U-shaped trapping site with a diameter of 100 μm; FIG. 7C illustrates a side view of the exemplary device, which includes a 250 μm flow channel layer situated on the substrate below the microwell array; FIG. 7D portrays the primary view of the exemplary device, which comprises a 1.1 mm thick cover, a microwell array slab, and a substrate that is 1 mm in thickness with a 0.25 mm spacer. While particular dimensions are used in this particular embodiments, it is appreciated that in other embodiments various other dimensions could be used. In some embodiments, the device can include alternative dimensions within a range, such as within +/−25% of the dimensions depicted in FIGS. 7A-7D. It is appreciated that the microwell concepts described herein are not limited to the dimensions and ranges shown and could be used in any numbers of different devices of varying sizes and dimensions.

FIG. 8 presents a schematic illustration of a multi-step reaction process 800 via sequential droplet addition facilitated by an exemplary microwell 801: both larger droplets Ld and smaller droplets Sd are dispersed throughout the flow channels and transported to the microwell 801 based on their respective sizes; the second (smaller) droplets Sd are absorbed or merged into the first (larger) droplet Ld, freeing up the U-shaped wells for subsequent smaller droplets Sd′, Sd″ to occupy and merge sequentially, thereby supporting multiple reactions In some embodiments, subsequent droplets of different batches can contain differing reagents. As shown, the larger droplets Ld and smaller droplets Sd are dispersed in the fluid in the fluid flow channels and are trapped within the microwell 801 after which the droplets merge into Ld′. Additional smaller droplets Sd′ are dispersed in the fluid flow (same or different than the previous smaller droplets Sd) and are trapped in the microwell after which the droplets merge again into Ld″. Still further smaller droplets Sd″ are dispersed in the fluid flow (same or different than the previous smaller droplets Sd, Sd′) and are trapped in the microwell after which the droplets merge again into Ld′″. Thus, unlike prior conventional approaches, this configuration and process supports performing multi-step reactions in a robust and efficient manner.

FIGS. 9A-9H exhibit a step-by-step schematic (top view) and images (top view) of a multi-step reaction process via sequential droplet addition in a microwell array chip 900 having an microarray 901 with microwells 903 defined therein; FIG. 9A depicts the microfluidic chip 900 with microwell arrays; FIG. 9B illustrates the capture of larger droplets Ld (e.g. with diameters of 128 μm) in the circular trapping sites of the microwells;

FIG. 9C shows the capture of the first smaller droplets Sd (e.g. with a diameter of 83 μm) in the U-shape trapping sites; FIG. 9D displays the schematic and image of the microwells after the merging of the larger and smaller droplets, where the smaller droplet is absorbed into the larger droplet, leaving the U-shape microwell empty for the subsequent merging step; FIG. 9E demonstrates the occupation of the second batch of smaller droplets in the empty U-shape; FIG. 9F shows the second merging step, which evacuates the smaller trapping sites; FIG. 9G displays the trapping of the third batch of smaller droplets with the larger droplets that absorbed two batches of smaller droplets; and FIG. 9H shows the schematic of the droplets in the microwells after merging.

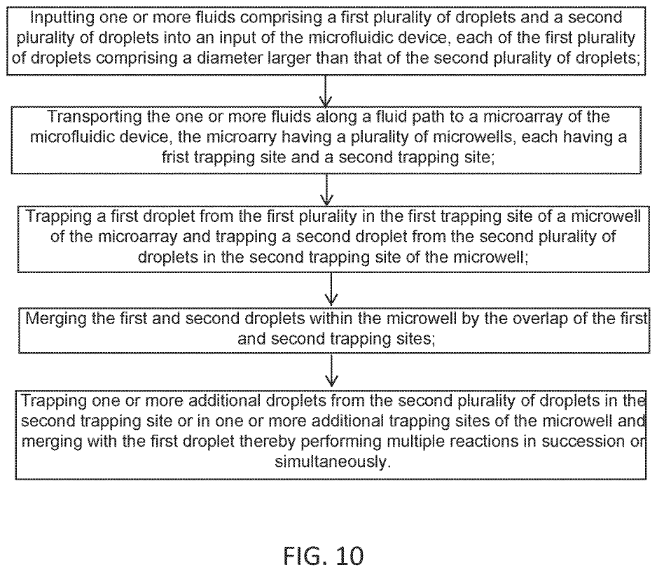

FIG. 10 shows a flowchart of an exemplary methods of performing multi-step reactions with a microfluidic device, as described in the embodiments above. The method includes steps of: inputting one or more fluids comprising a first plurality of droplets and a second plurality of droplets into an input of the microfluidic device, the first plurality of droplets comprising a diameter larger than that of the second plurality of droplets; transporting the one or more fluid paths along a fluid path to a microarray of the microfluidic device, the microarray having a plurality of microwells, each having a first trapping site and a second trapping site; trapping a first droplet from the first plurality in a first trapping site of a microwell within an microwell array of the microfluidic device, and trapping a second droplet from the second plurality of droplets in the second trapping site of the microwell within the microwell array of the microfluidic device; merging the first and second droplets within the microwell by the overlap of the first and second trapping sites; and trapping one or more additional droplets from the second plurality of droplets in the second trapping site or one or more additional trapping sites of the microwell and merging with the first droplet thereby performing multiple reactions in succession or simultaneously. The microfluidic array can be utilized microwells in accordance with any of the above, such as those in FIGS. 3A-3C.

While invention has been described with reference to specific embodiments, the invention is not limited thereto. Various features, embodiments and aspects of the above-described invention can be used individually or jointly. Further, the invention can be utilized in any number of environments and applications beyond those described herein without departing from the broader spirit and scope of the invention. The specification and drawings are illustrative rather than restrictive. The term “about” is +/−10%, or +/−20% of the recited value. It is recognized the terms “comprising,” “including,” and “having,” as used herein, are specifically intended to be read as open-ended terms of art. Any patent publications or references noted is incorporated herein by reference in their entirety for all purposes.

What is claimed is:

1. A microfluidic device comprising:
an input for receiving one or more fluids comprising a first plurality of droplets and a second plurality of droplets, the first plurality of droplets each having a larger diameter than that of the second plurality of droplets; and
a microarray having a plurality of wells, each well having a first trapping site configured for trapping a first droplet of the first plurality of droplets and at least a second trapping site configured for trapping a second droplet from the second plurality of droplets, wherein the first and second trapping sites are configured to trap larger droplets and smaller droplets, respectively, wherein a ratio of larger to smaller droplet diameter is within a range of about 1.5-1.9:1. and wherein the second trapping site overlaps with the first trapping site to facilitate merger of the second droplet with the first droplet to allow for multiple reactions either simultaneously or in succession.

2. The microfluidic device of claim 1, wherein the second trapping site is U-shaped so as to facilitate merger of the second droplet with the first droplet.

3. The microfluidic device of claim 2, where the first trapping site is substantially circular in shape.

4. The microfluidic device of claim 3, wherein the first trapping site has a diameter within a range from 100 to 600 μm, and the U-shaped trapping site has a diameter within a range from 57 to 400 μm.

5. The microfluidic device of claim 1, wherein the first and second trapping sites are of a same depth.

6. The microfluidic device of claim 1, where the first trapping site has a greater depth than that of the second trapping site.

7. The microfluidic device of claim 1, wherein the overlap is between 10-30% to facilitate merger of the second droplet with the first droplet.

8. The microfluidic device of claim 7, wherein the overlap is 20%.

9. The microfluidic device of claim 1, wherein the at least second trapping site comprises two trapping sites, each configured for trapping a second droplet from the second plurality of droplets and each overlapping with the first trapping site.

10. The microfluidic device of claim 1, wherein the at least second trapping site comprises three trapping sites, each configured for trapping a second droplet from the second plurality of droplets and each overlapping with the first trapping site.

11. The microfluidic device of claim 1, wherein the at least second trapping site comprises four trapping sites, each configured for trapping a second droplet from the second plurality of droplets and each overlapping with the first trapping site.

12. The microfluidic device of claim 1, wherein the first plurality of droplets comprises biological cells, and optionally the second plurality of droplets comprises one or more reagents.

13. The microfluidic device of claim 13, wherein the second plurality of droplets comprises biological cells, and optionally the first plurality of droplets comprises one or more reagents.

14. The microfluidic device of claim 1, wherein the microarray is defined in a Polydimethylsiloxane (PDMS) chip.

15. The microfluidic device of claim 14, wherein the PDMS chip is disposed between a transparent cover and a substrate, the substrate having one or more channel defined therein to at least partly define a flow channel from an input of the microfluidic device to the microarray.

16. A method of performing multiple reactions in succession or simultaneously using a microfluidic device, the method comprising:
inputting one or more fluids comprising a first plurality of droplets and a second plurality of droplets into an input of the microfluidic device, each of the first plurality of droplets comprising a diameter larger than that of the second plurality of droplets;
transporting the one or more fluids along a fluid path to a microarray of the microfluidic device, the microarray having a plurality of microwells, each having a first trapping site and a second trapping site;
trapping a first droplet from the first plurality of droplets in the first trapping site of the microwell;
trapping a second droplet from the second plurality of droplets in the second trapping site of the microwell;
merging the first and second droplets within the microwell by the overlap of the first and second trapping sites; and
trapping one or more additional droplets from the second plurality of droplets in the second trapping site or in one or more additional trapping sites of the microwell and merging with the first droplet thereby performing multiple reactions in succession or simultaneously.

17. The method of performing multiple reactions of claim 16, wherein each microwell is configured such that the first trapping site is circular and the second trapping site is U-shaped and overlaps with the first trapping site.

18. The method of performing multiple reactions of claim 16, wherein each microwell is configured with only the first and second trapping site such that the multiple reactions are performed sequentially.

19. The method of performing multiple reactions of claim 16, further comprising:
conducting incubation in the time between trapping the first and second droplets and merging the first and second droplets.

20. The method of performing multiple reactions of claim 16, wherein the one or more additional trapping sites are each U-shaped and overlap with the first trapping site to facilitate simultaneous reactions of the first droplet with the one or more additional droplets from the additional trapping sites.

21. The method of performing multiple reactions of claim 16, wherein trapping the first droplet from the first plurality of droplets in the first trapping site comprises tilting the microfluidics device.

22. The method of performing multiple reactions of claim 21, wherein trapping the second droplet from the second plurality of droplets in the second trapping site comprises tilting the microfluidics device.

23. The method of performing multiple reactions of claim 16, further comprising:
trapping a third droplet from a third plurality of droplets in the fluid in the second trapping site or in one or more additional trapping sites.

24. The method of performing multiple reactions of claim 16, wherein the first plurality of droplets comprises biological cells.

25. The method of performing multiple reactions of claim 16, wherein the second plurality of droplets comprises one or more reagents.
